(19) United States
(12) Patent Application Publication
(10) Pub. No.: US 2010/0028328 A1
(43) Pub. Date: Feb. 4, 2010

(54) TOPICAL TREATMENT FOR DISEASES OF EYE SURFACE

(75) Inventors: Andreas Reiff, San Marino, CA (US); Scott Hampton, Cumming, GA (US)

Correspondence Address: WILSON, SONSINI, GOODRICH & ROSATI, 650 PAGE MILL ROAD, PALO ALTO, CA, 94304-1050, US

(73) Assignee: THERAKINE, LTD., Cumming, GA (US)

(21) Appl. No.: 12/304,381

(22) PCT Filed: Jun. 12, 2006
(86) PCT No.: PCT/US06/22747

§ 371 Date: Jun. 30, 2009

(51) Int. Cl. A61K 39/395; A61K 48/00; A61K 38/00; A61P 27/02

(52) U.S. Cl. 424/130.1; 514/44 R; 514/2

(57) ABSTRACT

A topical eye solution for the treatment of corneal, scleral, or conjunctival diseases. In one embodiment of the present invention, the eye solution comprises a saline-based fluid, and an effective amount of at least one therapeutic compound or agent, wherein when at least one drop of the eye solution is applied to the surface of an eye, the therapeutic compound or agent is released to the cornea and conjunctiva of the eye.

This application is being filed as PCT International Patent application in the name of Therakine Limited, an Irish Corporation, Applicant for all countries except the U.S., and Andreas Reiff, a citizen of Germany, and Scott M. Hampton, a citizen of the U.S.A., Applicants for the designation of the U.S. only, on 8 Jun. 2006.

Some references, which may include patents, patent applications and various publications, are cited in a reference list and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [5] represents the 5th reference cited in the reference list, namely, Wilson S E. Molecular cell biology for the refractive corneal surgeon: programmed cell death and wound healing. Journal of Refractive Surgery 1997; 13(2): 171-5.

### FIELD OF THE INVENTION

The present invention is generally related to an eye solution, and more particularly, to a topical eye solution having at least one compound or agent which modulates inflammation related to a variety of diseases of the surface of the eye.

### BACKGROUND OF THE INVENTION

Inflammation of eye tissue can be caused by disease, injury, or surgery, which needs to be managed or treated. However, an eye solution that can be applied topically to a desired area of an eye without disturbing other parts of the eye and also effectively manage inflammation of eye tissue is lacking.

Therefore, a heretofore unaddressed need exists in the art to address the aforementioned deficiencies and inadequacies.

### SUMMARY OF THE INVENTION

In one aspect, the present invention relates to an eye solution. In one embodiment of the present invention, the eye solution comprises a saline-based fluid, and an effective amount of at least one therapeutic compound or agent, wherein when at least one drop of the eye solution is applied to the surface of an eye, the therapeutic compound or agent is released to the cornea and conjunctiva of the eye.

The eye solution may further comprise a gelling or thickening agent, wherein the gelling or thickening agent is one of polyvinyl alcohol, methyl cellulose, carbapol, or hyaluronic acid. In one embodiment of the present invention, the at least one therapeutic compound or agent comprises one or more biologic or small molecule modulators of the action of Interferon gamma (IFNg), Tumor Necrosis Factor alpha (TNFa), and the Merleukines including Interleukine-1,Interleukine-2, Interleukine-4, Interleukine-6, Interleukine-8, Interleukine-12, Interleukine-15, Interleukine-17, and Interleukine-18. The at least one therapeutic compound or agent may also comprise a biologic compound.

In another embodiment of the present invention, the at least one therapeutic compound or agent comprises at least a first and a second therapeutic compounds, at least one of the first and the second therapeutic compounds is an anti-cytokine or anti-chemokine for the treatment of inflammatory diseases In yet another embodiment of the present invention, the at least one therapeutic compound or agent comprises at least one of antibodies, nanobodies, antibody fragments, signaling pathway inhibitors, transcription factor inhibitors, receptor antagonists, small molecule inhibitors, oligonucleotides, fusion proteins, peptides, protein fragments, allosteric modulators of cell surface receptors including G-protein coupled receptors (GPCR), cell surface receptor internalization inducers, and GPCR inverse agonists.

In a further embodiment of the present invention, the at least one therapeutic compound or agent comprises at least one of the following small molecules that inhibit or block at least one of the intracellular signaling pathways, or regulatory enzymes/kinases of PTEN, PB Kinases, P38 MAP Kinase and MAP Kinases, all stress activated protein kinases (SAPKs), the ERK signaling pathways, the JNK signaling pathways (JNK1, JNK2), all RAS activated pathways, all Rho mediated pathways, the pathways NIK, MEKK-1, IKK-1, IKK-2, and intracellular and extracellular signaling pathways.

In use, at least one drop of the eye solution is applied on the exterior of the eye. Additional drops can also be applied to the exterior of the eye. The eye solution can also be used in the form of a gel so that an effective amount of at least one therapeutic compound or agent is delivered to the surface of the eye, the cornea and the conjunctiva, in the form of a topical drop or gel.

In another aspect, the present invention relates to inflammation control of eye tissue by selective use of pathway modulators, promoters, or inhibitors, such that undesirable inflammation can be suppresses or eliminated.

These and other aspects of the present invention will become apparent from the following description of the preferred embodiment taken in conjunction with the following drawings, although variations and modifications therein may be affected without departing from the spirit and scope of the novel concepts of the disclosure.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### DEFINITIONS

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification. Furthermore, subtitles may be used to help a reader of the specification to read through the specification, which the usage of subtitles, however, has no influence on the scope of the invention.

As used herein, “around”, “about” or “approximately” shall generally mean within 20percent, preferably within 10 percent, and more preferably within 5 percent of a given value or range. Numerical quantities given herein are approximate, meaning that the term “around”, “about” or “approximately” can be inferred if not expressly stated.

As used, the term “LVC” or “Laser Vision Correction”, is referred generally to any of the methods of correction of refractive visual error in which a laser is used to ablate tissue from the cornea to change the optical performance. Examples of this method are LASIK, PRK, and epi-LASIK.

The term “compound” is referred to a chemical combination of two or more elements that may have an impact on any living system such as a cell, nerve or tissue. Examples of compounds that may be related to practicing the present invention include those in the following exemplary list:

Anti-inflammatory compounds and example of currently available agents suitable to the treatment of surface diseases of the eye:
a) Anti-cytokines
   Anti-Tumor Necrosis Factor alpha (TNFα) such as
      (1) Etanercept (p75 TNFr fusion protein)
      (2) Infliximab (chimeric Anti TNF Mab)
      (3) Adalimumab (human Anti TNF Mab)
      (4) Onercept (soluble ρ55 TNFr)
      or other compounds, such as antibodies, nanobodies, antibody fragments, and receptor antagonists.
   Anti-Interleukin-1 such as
      (1) Anakinra (IL-1 type 1 receptor antagonist)
      (2) IL1 Trap (Regeneron, an IL-1 type 1 receptor plus IL-1 fusion protein)
      or other compounds
   Anti-interleukin-2 such as
      (1) Daclizumab or other compounds
   Anti-interleukin-4 such as
      (1) Human Anti-IL-4 antibody, E coli derived goat IgG (R&D systems)
      (2) Human Anti-IL-4 antibody, E coli derived murine IgG (R&D systems)
      Or other compounds
   Anti-interleukin-6 such as
      (1) MRA (Chugai Pharmaceuticals/Roche) or other compounds
   Anti-Interleukin-8 such as (1) Anti-EGF-R antibody (C225) or other compounds
   Anti-interleukin-12 such as
      (1) Human Anti-IL-12 antibody, E coli derived goat IgG (R&D systems)
      (2) Human Anti-IL-12 antibody, E coli derived murine IgG (R&D systems)
      Or other compounds
   Anti-Interleukin-15 such as
      (1) Human Anti-IL-15 antibody, E coli derived goat IgG (R&D systems)
      (2) Human Anti-IL-15 antibody, E coli derived murine IgG (R&D systems)
      Or other compounds
   Anti-Interleukin-17 such as
      (1) Human Anti-IL-17 antibody, E coli derived goat IgG (R&D systems)
      (2) Human Anti-IL-17 antibody, E coli derived murine IgG (R&D systems)
      Or other compounds
   Anti-Interleukin-18 such as
      (1) Human Anti-IL-18 antibody, E coli derived goat IgG (R&D systems)
      (2) Human Anti-IL-18 antibody, E coli derived murine IgG (R&D systems)
      Or other compounds
b) Cytokines
   Interleukin 10 and 12
c) Small molecule inhibitors that inhibit or block the following intracellular signaling pathways, or regulatory enzymes/kinases, for example:
   PTEN
   PB Kinases
   P38 MAP Kinase and other MAP Kinases
   AU stress activated protein kinases (SAPKs)
   The ERK signaling pathways
   The JNK signaling pathways (JNK1, JNK2)
   All RAS activated pathways
   AU Rho mediated pathways
   NTK, MEKK-1, UCK-1, IKK-2.

The term “agent” is broadly defined as anything that may have an impact on any living system such as a cell, nerve or tissue. For examples, the agent can be a chemical agent. The agent can also be a biological agent. The agent may comprise at least one known component. The agent can also be a physical agent. Other examples of agent include biological warfare agents, chemical warfare agents, bacterial agents, viral agents, other pathogenic microorganisms, emerging or engineered threat agents, acutely toxic industrial chemicals (TICS), toxic industrial materials (TIMS) and the like. Preferably, biological or pharmacological agents are employed to practice the present invention. Examples of agent types that may be related to practicing the present invention include antibodies, nanobodies, antibody fragments, signaling pathway inhibitors, transcription factor inhibitors, receptor antagonists, small molecule inhibitors, oligonucleotidesoligonucleotides, fusion proteins, peptides, protein fragments, allosteric modulators of cell surface receptors such as G-protein coupled receptors (GPCR), cell surface receptor internalization inducers, and GPCR inverse agonists.

The term “saline” is referred to a biocompatible physiological solution of sodium chloride (NaCl) in water at a concentration such that it is equivalent in concentration to human tears. Saline may be buffered with a number of compounds to maintain correct pH, and may include a variety of agent for thickening or improving adhesion and retention on the surface of the eye, such as poly-vinyl alcohol or methyl cellulose, for example. de

### DESCRIPTION OF THE PREFERRED EMBODIMENTS

Among other things, the present invention relates to the treatment of corneal inflammation subsequent to, or caused by, surgery, infection, injury, or other disease. The cornea has unique anatomic, cellular, molecular, and functional features that lead to important mechanistic differences in the process of repair in comparison with what occurs in skin and other organs. Corneal epithelial and stromal wound healing after injuries or intentional trauma such as refractive surgery is a complex process wherein the severity of apoptosis and reactivation of keratocytes is closely correlated with haze formation, corneal edema, neovascularization, and opacity [1-3]. Penetrating injuries typically heal by deposition of fibrotic “repair tissue” that fills and seals wounds but does not restore normal function. Collagen degradation by corneal fibroblasts is an underlying cause of corneal ulceration and excessive deposition of fibrotic repair tissue can lead to excessive scarring and corneal contracture. In the cornea, fibrotic repair presents special challenges affecting both clarity and shape of the cornea, which is an essential component of the ability of the eye to focus [4, 5].

On the other hand with increasing popularity of surgical refractive correctional vision surgery using various laser techniques that alter corneal refractive errors, understanding of cornea repair mechanisms has gained increasing importance. Here haze formation, corneal edema, neovascularization, and opacity are unwanted complications and are a major determinant of safety and efficacy influencing visual outcome [6-10]. LASIK and PRK are the most common refractive procedures; however, alternative techniques, including LASEK, PRK with mitomycin C, and Epi-LASIK. have been developed in an attempt to overcome common complications. Clinical outcomes and a number of common complications are directly related to the healing process and the unpredictable nature of the associated corneal cellular response. These complications include overcorrection, undercorrection, regression, corneal stroma opacification, and many other side effects that are based on the biologic response to surgery [3,10].

Postoperative histological studies demonstrate that the corneal healing process consists of corneal epithelial and endothelial cell loss and a large amount of inflammatory cell infiltration into the corneal stroma [11-13]. Most of the infiltrating cells are neutrophils, lymphocytes and macrophages. In the inflamed cornea, neutrophils might be responsible for acute corneal edema/opacity and macrophages for corneal angiogenesis and chronic inflammation [14-16].

The corneal epithelium, stroma, nerves, inflammatory cells, and lacrimal glands are the main tissues and organs involved in the wound healing response to corneal surgical procedures. Complex cellular interactions mediated by cytokines and growth factors occur among the cells of the cornea, resulting in highly variable biologic responses. Among the best-characterized processes are keratocyte apoptosis, keratocyte necrosis, keratocyte proliferation, migration of inflammatory cells with subsequent cytokine release, and myofibroblast generation. These cellular interactions are involved in extracellular matrix reorganization, stromal remodeling, wound contraction, and several other responses to surgical injury [17]. Therefore a better understanding of the complete cascade of events involved in the corneal wound healing process and anomalies that lead to complications is critical to improve the efficacy and safety of refractive surgical procedures.

Recent advances in understanding the biologic and molecular processes that contribute to the healing response demonstrated that inflammation and corneal wound healing is highly associated with increased cytokine levels especially IL-1. The IL-1 alpha feedback loop is an important mechanism by which fibroblasts adopt a repair phenotype during remodeling of the cornea. Moreover mechanical trauma to the mouse cornea triggers the enhanced synthesis of IL-1 alpha and IL-IR, which in turn results in the production of IL-6 and more IL-1 alpha. [18-20]. At the intracellular level IL-lbeta-induced phosphorylation of the MAPKs extracellular signal-regulated kinase (ERK) and c-Jun N-terminal kinase (INK), but not that of p38contribute to the inflammatory response and delayed wound healing [19]. In addition to anti-inflammation, II-1 antagonists inhibit proliferation of fibroblast-like corneal and conjunctival cells, indicating that these compounds could be used not only as anti-uveitis agents but also as useful agents to prolong the functional period of aqueous humor outflow after trabeculectomy [21].

Supporting evidence of this observation comes from the treatment of Herpetic Stromal Keratitis (HSK), a T cell-orchestrated, immunoinflammatory Herpes simplex virus infection of the cornea. Following ocular HSV-1 infection, neovascularization of the avascular cornea is a critical event in the pathogenesis of herpetic stromal keratitis [22]. It has been well demonstrated that IL-1 and IL-6 play an important role in the pathogenesis of HSK [23]. IL-6 produced from virus-infected cells can stimulate noninfected resident corneal cells and other inflammatory cells to secrete VEGF, a potent angiogenic factor [23]. Treatment of HSK with a receptor antagonist to IL-1 (IL-1 ra) has been shown to reduce the influx of cells of the innate and adaptive immune system into the cornea, hi addition, treatment with an anti-IL-6 agent diminished corneal vascular endothelial growth factor levels, resulting in reduced corneal angiogenesis [21]. This further demonstrates the close relationship between proinflammatory cytokines and VEGF-induced corneal neovascularization. In addition to II-1 and 11-6, a multitude of other cytokines, chemokines and metalloproteinases are involved in corneal wound healing [24]. For example TGF-beta 1 and VEGF are components of normal tear fluid and are significantly increased after excimer laser photorefractive keratectomy indicating that they may influence the corneal wound healing process [25]. Especially TGF-beta 1 is responsible for recruitment of activated keratocytes, myofibroblast transformation, and stromal fibrosis [26] Therefore treatment with anti-TGFbeta and anti-VEGF may be useful even though only in a limited form to reduce post-PRK corneal haze development in patients [27]. Furthermore TNF-alpha, also a component of normal tear fluid has been found to be significantly increased during the postoperative days following PRK, suggesting a role in corneal wound healing [28]. An upregulation of the proinflammatory cytokines TNF-alpha and IL-6 modify the production of metalloproteinases in the corresponding cells resulting in collagenolytic corneal damage.

However in spite of hypersecretion caused by the corneal wound, TNF-alpha concentrations in the tear fluid remain constant during wound healing.

Multiple topical and systemic immunosuppressive agents such as glucorticoids or cyclosporme have been used in order to influence corneal wound healing by either suppressing collagen deposition and scarring or promoting anti-inflammatory mechanisms [29-33]. However data in animal models of corneal injury suggest that anti-cytokine inhibitors mainly targeting IL-1, 11-6 and TNF may be far more potent and effective than traditional immunosuppressants.

For example, monoclonal antibodies to TNF have been found effective in arresting progressive rheumatoid arthritis-associated peripheral ulcerative keratitis refractory to conventional immunomodulatory therapy [34], Lastly topical treatment with a TNF antagonist such as TNFR-I promoted the acceptance of allogeneic corneal transplants and inhibited gene expression of 2 chemokines (RANTES and macrophage inflammatory protein-lbeta) associated with corneal graft rejection [35]. This further supports the feasibility of a topical anticytokine strategy as a means of corneal wound healing and reducing corneal allograft rejection without resorting to the use of potentially toxic immunosuppressive drugs.

The present invention provides a different approach and offers a viable and superior treatment solution for inflammation of the surface tissues of the eye.

Thus, among other things, the present invention allows delivery of compounds or agents, such as monoclonal antibodies or kinase inhibitors, directly to the cornea and conjunctiva in the form of an topically applicable eye drop that contains at least one anti-inflammatory compound in a solution or gel.

In another embodiment, the eye drop will be buffered and contain at least one anti-inflammatory agent or compound.

In another embodiment, the eye drop may contain a thickening or gelling agent such as methyl cellulose, poly-vinyl alcohol, carbopol, or other biocompatible materials.

In one embodiment, the at least one anti-inflammatory agent or compound has one or more biologic or small molecule modulators of the action of Tumor Necrosis Factor alpha (TNFa); the Interleukines including Interleukine-1, Interleukine-2, Interleukine-4, Interleukine-6, Interleukine-8, Interleukine-12, Interleukine-15, Interleukine-17, and Interleukine-18.

in one embodiment, the agent or compound is one or more monoclonal antibody, nanobody, antibody fragments, signaling pathway inhibitors, transcription factor inhibitors, traps, receptor antagonists, small molecule inhibitors, oligonucleotides, fusion proteins, peptides, protein fragments, allosteric modulators of cell surface receptors such as G-protein coupled receptors (GPCR), cell surface receptor internalization inducers, and GPCR inverse agonists.

In another embodiment, the agent or compound is one or both of IL-1O and IL-12.

In a preferred embodiment the agent or compound is one or more modulators of IL-1 or TNFa, in which the modulators are monoclonal antibodies, nanobodies, traps, or small molecules.

In another preferred embodiment the agent or compound is one or more small molecule inhibitors of an intracellular kinase such as PTEN, PD, or the MAP kinases.

The foregoing description of the exemplary embodiments of the invention has been presented only, for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

REFERENCES

1. Kuo I C. Corneal wound healing. [Review] [44 refs] Current Opinion in Ophthalmology 2004; 15(4): 311-5

2. Agrawal V B. Tsai R J. Corneal epithelial wound healing. [Review] [75 refs] Indian Journal of Ophthalmology 2003; 51(1): 5-15

3. Fini M E, Stramer B M. How the cornea heals: cornea-specific repair mechanisms affecting surgical outcomes. Cornea 2005 Nov; 24(8 Suppl): S2-S11

4. Wilson S. E. Everett. Kinsey Lecture. Keratocyte apoptosis in refractive surgery. CLAO Journal 1998; 24(3): 181-5

5. Wilson S E. Molecular cell biology for the refractive corneal surgeon: programmed cell death and wound healing. Journal of Refractive Surgery 1997; 13(2): 171-5

6. Netto M V, Mohan R R, Ambrosio R Jr, Hutcheon A E, Zieske J D, Wilson S E. Wound healing in the cornea: a review of refractive surgery complications and new prospects for therapy. Cornea. 2005 Jul; 24(5): 509-22

7. Netto M V. Wilson S E. Corneal wound healing relevance to wavefront guided laser treatments. [Review] [58 refs] Ophthalmology Clinics of North America 2004; 17(2): 225-31

8. Kuo I C. Lee S M. Hwang D G. Late-onset corneal haze and myopic regression after photorefractive keratectomy (PRK). Cornea 2004; 23(4): 350-5

9. Kaji Y. Yamashita H. Oshika T. Corneal wound healing after excimer laser keratectomy. [Review] [28 refs] Seminars in Ophthalmology 2003; 18(1): 11-6

10. Alio J L. Perez-Santonja J J. Tervo T. Tabbara K F. Vesaluoma M. Smith R J. Maddox B. Maloney R K. Postoperative inflammation, microbial complications, and wound healing following laser in situ keratomileusis. [Review] [82 refs] Journal of Refractive Surgery 2000; 16(5): 523-38

11. Dawson D G. Edelhauser H F. Grossniklaus H E. Long-term histopathologic findings in human corneal wounds after refractive surgical procedures. American Journal of Ophthalmology 2005 139(1): 168-78

12. Wachtlin J. Langenbeck K. Schrunder S. Zhang E P. Hoffmann F. Immunohistology of corneal wound healing after photorefractive keratectomy and laser in situ keratomileusis. Journal of Refractive Surgery 1999; 15(4): 451-8

13. Kato T. Nakayasu K. Hosoda Y. Watanabe Y. Kanai A. Corneal wound healing following laser in situ keratomileusis (LASIK): a histopathological study in rabbits. British Journal of Ophthalmology 1999; 83(11): 1302-5

14. Carlson E C, Drazba J, Yang X, Perez V L. Visualization and characterization of inflammatory cell recruitment and migration through the corneal stroma in endotoxin-induced keratitis. Invest Ophthalmol Vis Sci. 2006; 47(1): 241-8

15. Sonoda K H, Nakao S, Nakamura T, Oshima T, Qiao H, Hisatomi T, Kinoshita S, Ishibashi T. Cellular events in the normal and inflamed cornea. Cornea. 2005 Nov; 24(8 Suppl): S50-S54

16. O'Brien T P. Li Q. Ashraf M F. Matteson D M. Stark W J. Chan C C. Inflammatory response in the early stages of wound healing after excimer laser keratectomy. Archives of Ophthalmology 1998; 116(11): 1470-4 17. Ahmadi A J. Jakobiec F A. Corneal wound healing: cytokines and extracellular matrix proteins. [Review] [24 refs] International Ophthalmology Clinics 2002; 42(3): 13-22

18. Wilson S E. Mohan R R. Mohan R R. Ambrosio R Jr. Hong J. Lee J. The corneal wound healing response: cytokine-mediated interaction of the epithelium, stroma, and inflammatory cells. [Review] [58 refs] Progress in Retinal & Eye Research 2001; 20(5): 625-37

19. Narayanan S, Glasser A, Hu Y S, McDermott A M. The effect of interleukin-1 on cytokine gene expression by human corneal epithelial cells. Exp Eye Res. 2005; 80(2): 175-83. 20. Lu Y, Fukuda K, Liu Y, Kumara N, Nishida T. Dexamethasone inhibition of IL-I-induced collagen degradation by corneal fibroblasts in three-dimensional culture. Invest Ophthalmol Vis Sci. 2004; 45(9): 2998-3004

21. Biswas P S, Banerjee K, Zheng M, Rouse B T. Counteracting corneal immunoinflammatory lesion with interleukin-1 receptor antagonist protein. J Leukoc Biol. 2004; 76(4): 868-75

22. Li H, Zhang J, Kumar A, Zheng M, Atherton S S, Yu F S. Herpes simplex virus 1infection induces the expression of proinflammatory cytokines, interferons and TLR7 in human corneal epithelial cells. Immunology 2006 February; 117(2): 167-76

23. Biswas P S, Banerjee K, Kinchington P R, Rouse B T. Involvement of IL-6 in the paracrine production of VEGF in ocular HSV-I infection. Exp Eye Res. 2006; 82(1): 46-54

24. Malecaze F. Simorre V. Chollet P. Tack J L. Muraine M. Le Guellec D. Vita N. Arne J L. Darbon J M. Interleukin-6 in tear fluid after photorefractive keratectomy and its effects on keratocytes in culture. Cornea 1997; 16(5): 580-7 25. Vesaluoma M. Teppo A M. Gronhagen-Riska C. Tervo T. Release of TGF-beta

1 and VEGF in tears following photorefractive keratectomy. Current Eye Research 1997; 16(1): 19-25

26. Mita T, Yamashita H, Kaji Y, Obata H, Hanyu K, Suzuki M, Tobari I. Functional difference of TGF-beta isoforms regulating corneal wound healing after excimer laser keratectomy. Exp. Eye Res. 1999; 68:513-519

27. Moller-Pedersen T. Cavanaugh H D. Petroll W M. Jester J V. Neutralizing antibody to TGFbeta modulates stromal fibrosis but not regression of photoablative effect following PRK. Current Eye Research 1998. 17(7): 736-47 28. Vesaluoma M. Teppo A M, Gronhagen-Riska C. Tervo T. Increased release of tumour necrosis factor-alpha in human tear fluid after excimer laser induced corneal wound. British Journal of Ophthalmology 1997; 81(2): 145-9 29. Kim T I, Lee S Y, Pak J H, Tchah H, Kook M S. Mitomycin C, ceramide, and 5-fluorouracil inhibit corneal haze and apoptosis after PRK. Cornea. 2006; 25(1): 55-60

30. Kaji Y. Amano S. Oshika T. Obata H. Ohashi T. Sakai H. Shirasawa E. Tsuru T. Yamashita H. Effect of anti-inflammatory agents on corneal wound-healing process after surface excimer laser keratectomy. Journal of Cataract & Refractive Surgery 2000; 26(3): 426-31 31. Assouline M. Renard G. Arne J L. David T. Lasmolles C. Malecaze F.

Pouliquen Y J. A prospective randomized trial of topical soluble 0.1% indomethacin versus 0.1% diclofenac versus placebo for the control of pain following excimer laser photorefractive keratectomy. Ophthalmic Surgery & Lasers 1998; 29(5): 365-74 32. Chang J H, Kook M C, Lee J H, Chung H, Wee W R. Effects of synthetic inhibitor of metalloproteinase and cyclosporine A on corneal haze after excimer laser photorefractive keratectomy in rabbits. Exp Eye Res. 1998; 66(4): 389-96

33. Filipec M, Phan T M, Zhao T Z, Rice B A, Merchant A, Foster C S. Topical cyclosporine A and corneal wound healing. Cornea. 1992; 11(6): 546-52.

34. Thomas J W, Pflugfelder S C. Therapy of progressive rheumatoid arthritis-associated corneal ulceration with infliximab. Cornea. 2005 Aug; 24(6): 742-4

35. Qian Y. Dekaris I. Yamagami S. Dana M R. Topical soluble tumor necrosis factor receptor type I suppresses ocular chemokine gene expression and rejection of allogeneic corneal transplants. Archives of Ophthalmology 2000; 118(12): 1666-71

What is claimed is:

1. An eye solution, comprising: a. a saline-based fluid; and b. an effective amount of at least one therapeutic compound or agent, wherein when at least one drop of the eye solution is applied to the surface of an eye, the therapeutic compound or agent is released to the cornea and conjunctiva of the eye.

2. The eye solution of claim 1, further comprising a gelling or thickening agent.

3. The eye solution of claim 2, wherein the gelling or thickening agent is one of polyvinyl alcohol, methyl cellulose, carbapol, or hyaluronic acid.

4. The eye solution of claim 1, wherein the at least one therapeutic compound or agent comprises one or more biologic or small molecule modulators of the action of Interferon gamma (IFNg), Tumor Necrosis Factor alpha (TNFa), and the Interleukines including Inlerleukine-1, Interleukine-2, Interleukine-4, Interleukine-6, Interleukine-8, Interleukine-1 2, Interleukine-1 5, Interleukine-17, and Interleukine-18.

5. The eye solution of claim 1, wherein the at least one therapeutic compound or agent comprises a biologic compound.

6. The eye solution of claim 5, wherein the at least one therapeutic compound or agent comprises at least a first and a second therapeutic compounds, at least one of the first and the second therapeutic compounds is an anti-cytokine or anti-chemokine for the treatment of inflammatory diseases.

7. The eye solution of claim 5, wherein the at least one therapeutic compound or agent comprises at least one of antibodies, nanobodies, antibody fragments, signaling pathway inhibitors, transcription factor inhibitors, receptor antagonists, small molecule inhibitors, oligonucleotides, fusion proteins, peptides, protein fragments, allosteric modulators of cell surface receptors including G-protein coupled receptors (GPCR), cell surface receptor internalization inducers, and GPCR inverse agonists.

8. The eye solution of claim 1, wherein the at least one therapeutic compound or agent comprises at least one of the following small molecules that inhibit or block at least one of the intracellular signaling pathways, or regulatory enzymes/kinases of PTEN, PD Kinases, P38 MAP Kinase and MAP Kinases, all stress activated protein kinases (SAPKs), the ERK signaling pathways, the JNK signaling pathways (JNK1, JNK2), all RAS activated pathways, all Rho mediated pathways, the pathways NIK, MEKK-1, IKK-1, IKK-2, and intracellular and extracellular signaling pathways.

9. The eye solution of claim 1, wherein the at least one drop of the eye solution is applied on the exterior of the eye.
